(19) United States
(12) Patent Application Publication
(10) Pub. No.: US 2019/0241301 A1
(43) Pub. Date: Aug. 8, 2019

(54) PROCESS AND APPARATUS FOR LABELING METERED DOSE INHALER

(71) Applicant: Armstrong Pharmaceuticals Inc., Canton, MA (US)

(72) Inventors: Ping He, Sharon, MA (US); Lei Zhou, Canton, MA (US); Kevin Rego, Canton, MA (US)

(21) Appl. No.: 15/892,239

(22) Filed: Feb. 8, 2018

(51) Int. Cl. B65C 3/08; A61M 15/00

(52) U.S. Cl. CPC B65C 3/08; A61M 2207/10; A61M 15/009

(57) ABSTRACT

A labelling system configured to apply a label to a product container includes a conveyor having a movable track including an infeed station and an outfeed station opposite the infeed station. The labelling system also includes a spindle rotatably supported on the movable track configured to support the product container, an applicator arm configured to feed the label toward the conveyor when the movable track advances the spindle and the product container toward the applicator arm, an actuator configured to move between a retracted position and an extended position, and a roller rotatably coupled to an end of the actuator. The roller is configured to press a leading edge of the label against the product container when the actuator is in the extended position, and configured to rotate and follow along at least a portion of the product container and press the label against the portion of the product container.

### FIELD

The present disclosure relates generally to labelling systems.

### BACKGROUND

Pressure sensitive labels are commonly applied to product containers to display indicia (e.g., text, graphics, and/or symbols) for identifying the contents of the container, instructions for use, safety information, dosing instructions, and/or recycling information.

Conventional techniques for applying labels to product containers include manually applying the labels by hand or applying the labels with an automated system. However, labels applied to product container by these conventional techniques are prone wrinkling, bubbling, misalignment on the product container, and/or other defects, particularly when the product container has complex geometry, such as a non-cylindrical surfaces (e.g., a surface having rotational asymmetry).

### SUMMARY

The present disclosure is directed to various embodiments of a labelling system configured to apply a label to a product container. In one embodiment, the labelling system includes a conveyor including a movable track having an infeed station and an outfeed station opposite the infeed station, a spindle rotatably supported on the movable track and configured to support the product container, an applicator arm configured to feed the label toward the conveyor when the movable track advances the spindle and the product container toward the applicator arm, an actuator configured to move between a retracted position and an extended position, and a roller rotatably coupled to an end of the actuator. When the product container is supported on the spindle and the movable track is actuated to advance the spindle and the product container toward the applicator arm, the actuator moves into the extended position, the roller presses a leading edge of the label against the product container, and the roller rotates and follows along at least a portion of the product container and presses the label against the portion of the product container.

The actuator may be configured to swing in a first direction about an axis substantially perpendicular to the movable track when the product container contacts the roller.

The labelling system may include a spring hinge coupled to the actuator that is configured to bias the actuator to swing in a second direction opposite the first direction.

An outer surface of the roller may include a resilient material such as foam.

The actuator may be a hydraulic cylinder or a pneumatic cylinder.

The labelling system may include a pair of rails supported above the movable track and extending from a first end proximate to the infeed station of the conveyor and a second end proximate to the applicator arm.

The labelling system may include a peel plate proximate to an end of the applicator arm proximate to the conveyor. The peel plate is configured to peel the label off of a removable backing sheet.

The labelling system may include an anti-static bar proximate to the end of the applicator arm proximate to the conveyor.

A labelling system according to another embodiment of the present disclosure includes a conveyor including a movable track having an infeed station and an outfeed station opposite the infeed station, a spindle rotatably supported on the movable track including a spur gear and a support post configured to support the product container, an applicator arm configured to feed the label toward the conveyor when the movable track advances the spindle and the product container toward the applicator arm, an actuator configured to move between a retracted position and an extended position, a gear rack positioned along a side of the conveyor, and an applicator plate positioned along at least a portion of the gear rack including a resilient material facing a centerline of the conveyor. When the product container is supported on the support post of the spindle and the movable track is actuated to advance the spindle and the product container toward the applicator arm, the actuator moves into the extended position and presses a leading edge of the label against the product container, the spur gear engages the gear rack, which rotates the spindle and the product container, and the resilient material of the applicator plate presses a remaining portion of the label onto the product container as the spindle and the product container are rotating.

The resilient material of the applicator plate may include ethylene propylene diene monomer.

The gear rack may substantially straight and the applicator plate may be substantially planar.

The gear rack may be configured to rotate the spindle and the product container at least one complete revolution along the applicator plate.

The labelling system may include a roller rotatably coupled to an end of the actuator.

The labelling system may include a pair of rails supported above the movable track and extending from a first end proximate to the infeed station of the conveyor and a second end proximate to the applicator arm.

The labelling system may include a peel plate proximate to an end of the applicator arm proximate to the conveyor. The peel plate is configured to peel the label off of a removable backing sheet.

The labelling system may include an anti-static bar proximate to the end of the applicator arm proximate to the conveyor.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable device.

### BRIEF DESCRIPTION OF THE DRAWINGS

These and other features and advantages of embodiments of the present disclosure will become more apparent by reference to the following detailed description when considered in conjunction with the following drawings. In the drawings, like reference numerals are used throughout the figures to reference like features and components. The figures are not necessarily drawn to scale.

FIGS. 1A-1B are perspective views of a product container including a label that was applied to the product container by a labelling system of the present disclosure;

FIG. 2 is a schematic layout view of a labelling system according to one embodiment of the present disclosure;

FIGS. 3A-3B are a detail view and an exploded detail view showing a spindle assembly of the embodiment of the labelling system illustrated in FIG. 2;

FIGS. 4A-4C are detail views showing an actuator of the embodiment of the labelling system illustrated in FIG. 2 in a first angular position and a second angular position; and

FIGS. 5A-5B are detail views showing the spindle assembly engaging a gear rack of the embodiment of the labelling system illustrated in FIG. 2.

### DETAILED DESCRIPTION

The present disclosure is directed to various embodiments of a labelling system and a method for applying a pressure sensitive label to a product container, such as an actuator of a metered dose inhaler.

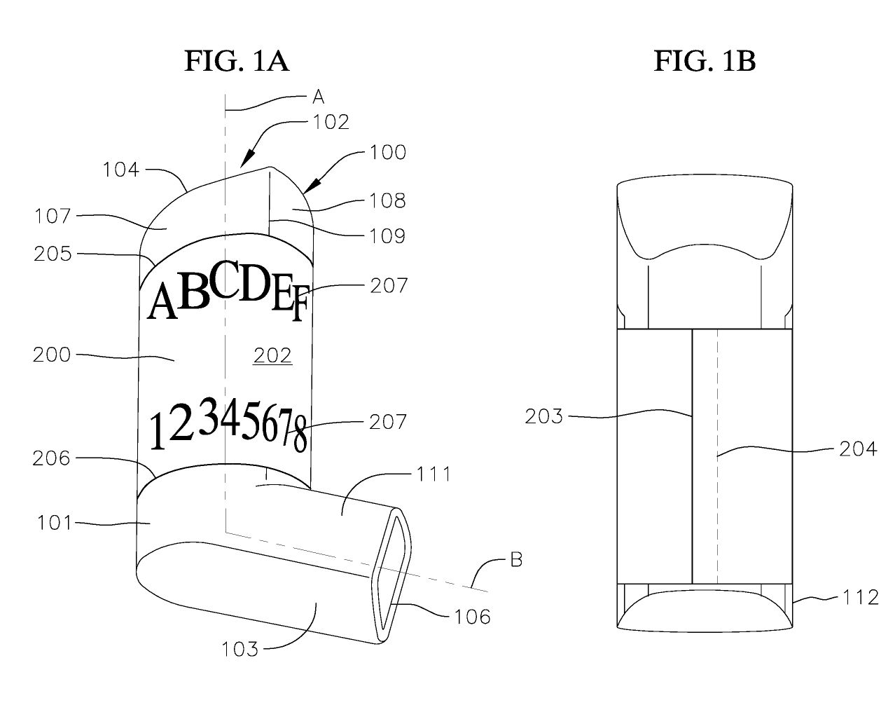

FIGS. 1A-1B are perspective views of a product container 100 including a pressure sensitive label 200 that was applied to the product container 100 by a labelling system 300 of the present disclosure. In the illustrated embodiment, the product container 100 is an actuator of a metered dose inhaler. In one or more embodiments, the labelling system 300 of the present disclosure may be utilized to apply the pressure sensitive labels 200 to any other type of product containers. In the illustrated embodiment, each of the actuators 100 of the metered dose inhalers includes an actuator body 101 defining a cavity 102 configured to accommodate at least a portion of a canister containing a medicament (e.g., a mixture of a propellant and a medicament), and a mouth piece 103 connected to the actuator body 101. The actuator body 101 also includes an opening 104 in communication with the cavity 102 configured to permit the canister to be inserted into the cavity 102. In the illustrated embodiment, the opening 104 and the mouth piece 103 are at opposite ends of the actuator body 101 (e.g., the opening 104 is at an upper end of the actuator body 101 and the mouth piece 103 is at a lower end of the actuator body 101). In the illustrated embodiment, a longitudinal axis A of the actuator body 101 defines an obtuse angle relative to a longitudinal axis B of the mouth piece 103, and an upper edge 105 of the actuator body 101 is perpendicular or substantially perpendicular to the longitudinal axis A of the actuator body 101. The mouth piece 103 of the actuator 100 includes an opening 106 through which the medicament in the canister is dispensed to a user. Additionally, in the illustrated embodiment, the actuator body 101 is non-cylindrical (e.g., the actuator body 101 is rotationally asymmetric) and includes a back surface 107 and a front surface 108 connected to the back surface 107 at a pair of edges 109, 110. In the illustrated embodiment, the back surface 107 has a relatively deep curvature and the front surface 108 has a relatively shallow curvature, although in one or more embodiments the actuator body may have any other suitable shape.

With continued reference to the embodiment illustrated in FIGS. 1A-1B, the pressure sensitive label 200 includes an adhesive surface 201 adhered to the back surface 107 and the front surface 108 of the actuator body 101, and a non-adhesive surface 202 opposite the adhesive surface 201 facing outward away from the front and back surfaces 107, 108 of the actuator body 101. In the illustrated embodiment, the pressure sensitive label 200 wraps entirely around the actuator body 101 (e.g., one end portion 203 of the pressure sensitive label 200 overlaps an opposite end portion 204 of the pressure sensitive label 200). In one or more embodiments, at least one of the end portions 203, 204 (e.g., a leading end portion 204) of the pressure sensitive label 200 is aligned or substantially aligned with a laterally centered portion of the back surface 107 of the actuator body 101. Additionally, in the illustrated embodiment, upper and lower edges 205, 206 of the pressure sensitive label 200 are parallel or substantially parallel to the upper edge 105 of the actuator body 101. The non-adhesive surface 202 of the pressure sensitive label 200 may include one or more indicia 207, such as text, symbols, and/or graphics. The content of the one or more indicia 207 may vary depending on the nature of the product container on which the pressure sensitive label 200 is applied. For instance, in one or more embodiments in which the product container 100 is an actuator of a metered dose inhaler, the indicia 207 may include usage instructions and/or warnings.

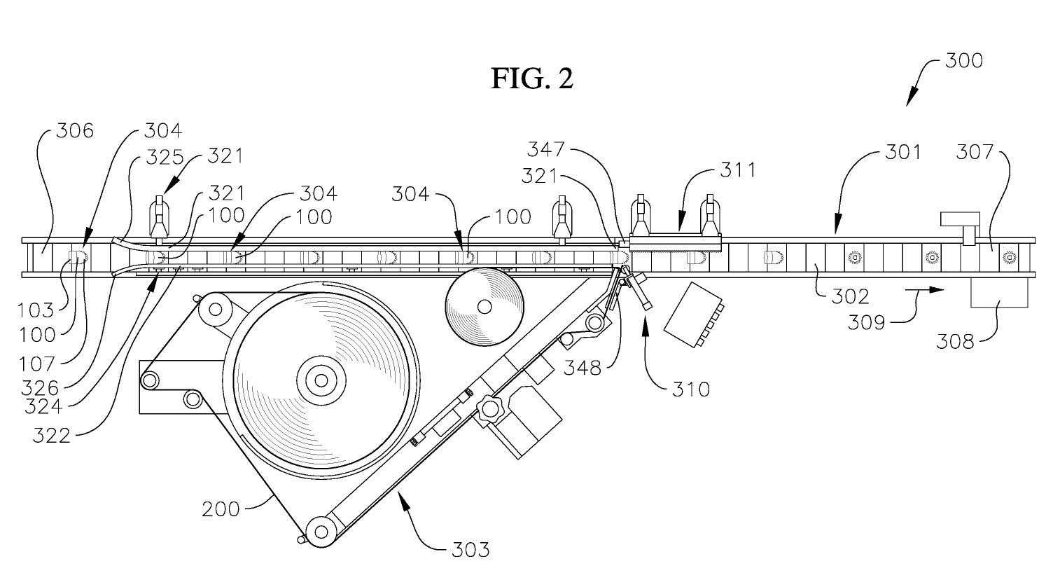

With reference now to FIG. 2, a labelling system 300 according to one embodiment of the present disclosure is configured to apply pressure sensitive labels to product containers. The labeling system 300 is described hereinafter with reference to applying the pressure sensitive label 200 to the actuator 100 of the metered dose inhaler, as illustrated in FIGS. 1A-1B, although in one or more embodiments the labelling system 300 may be utilized to apply labels to any other suitable type or kind of product container.

In the illustrated embodiment, the labelling system 300 includes a conveyor 301 including a movable conveyor track 302 (e.g., a conveyor belt), an applicator arm 303 positioned at one side of the conveyor 301, and a series of spindle assemblies 304 rotatably supported on the movable conveyor track 302. The spindle assemblies 304 are configured to support the actuators 100 of metered dose inhalers that are to be labeled by the labelling system 300. The applicator arm 303 is configured to unwind the labels 200 from a spool of labels supported on a label reel 305 positioned at or near an end of the applicator arm 303 distal to the conveyor 301, and feed the labels 200 toward the conveyor 301 for application onto the actuators 100 supported on the spindle assemblies 304. The conveyor 301 includes an infeed station 306 at which unlabeled actuators 100 are mounted onto the spindle assemblies 304 (e.g., manually loaded on the spindle assemblies 304), and an outfeed station 307 opposite to the infeed station 306 at which labeled actuators 100 (see FIGS. 1A-1B) may be unloaded from the spindle assemblies 304 of the labeling system 300. In the illustrated embodiment, a conveyor motor 308 coupled to the movable conveyor track 302 is configured to advance the movable conveyor track 302, the spindle assemblies 304 supported on the movable conveyor track 302, and the actuators 100 of the metered dose inhalers supported on the spindle assemblies 304 in a direction (arrow 309) from the infeed station 306 to the outfeed station 307.

In the illustrated embodiment, the labelling system 300 also includes an actuator assembly 310 configured to apply a leading edge portion of one of the labels 200 to each of the actuators 100 of the metered dose inhalers, and a wrapping assembly 311 configured to wrap a remaining portion of the label 200 around the actuator 100 of the metered dose inhaler. In the illustrated embodiment, the actuator assembly 310 is positioned proximate to an end of the applicator arm 303 proximate to the conveyor 301 and downstream of the applicator arm 303 (e.g., the actuator assembly 310 is positioned between the applicator arm 303 and the outfeed station 307 of the conveyor 301). Additionally, in the illustrated embodiment, the wrapping assembly 311 is positioned downstream of the applicator arm 303 and the actuator assembly 310 (e.g., the wrapping assembly 311 is positioned between the actuator assembly 310 and the outfeed station 307 of the conveyor 301). In the illustrated embodiment, the wrapping assembly 311 and the actuator assembly 310 are on opposite sides of the conveyor 301.

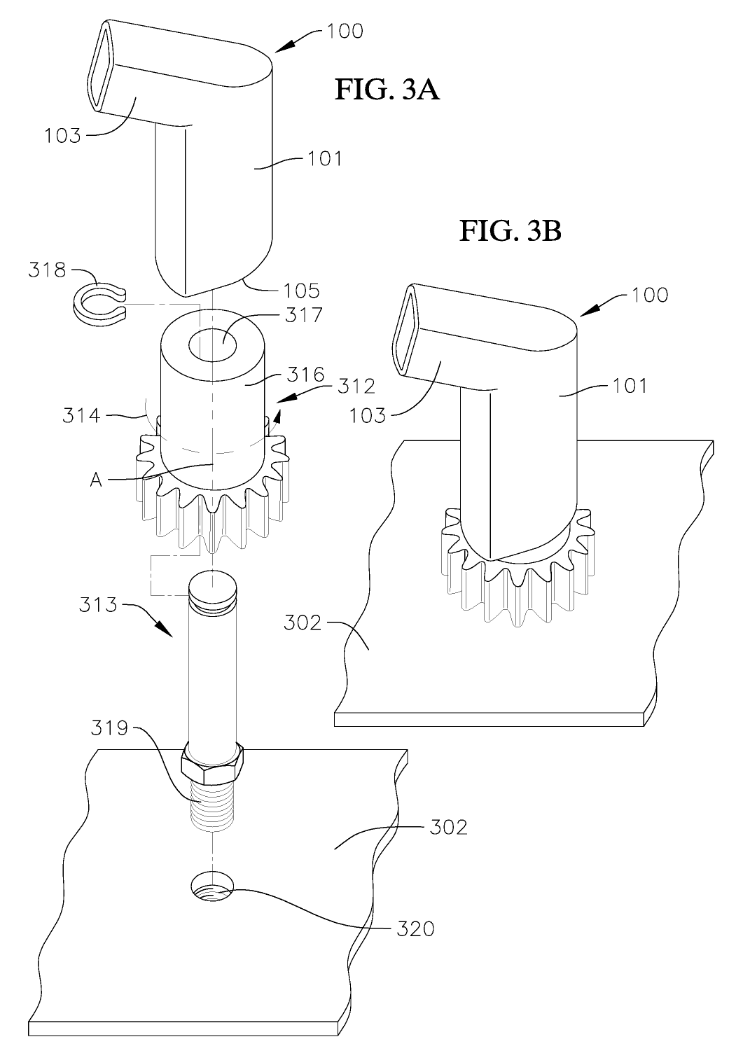

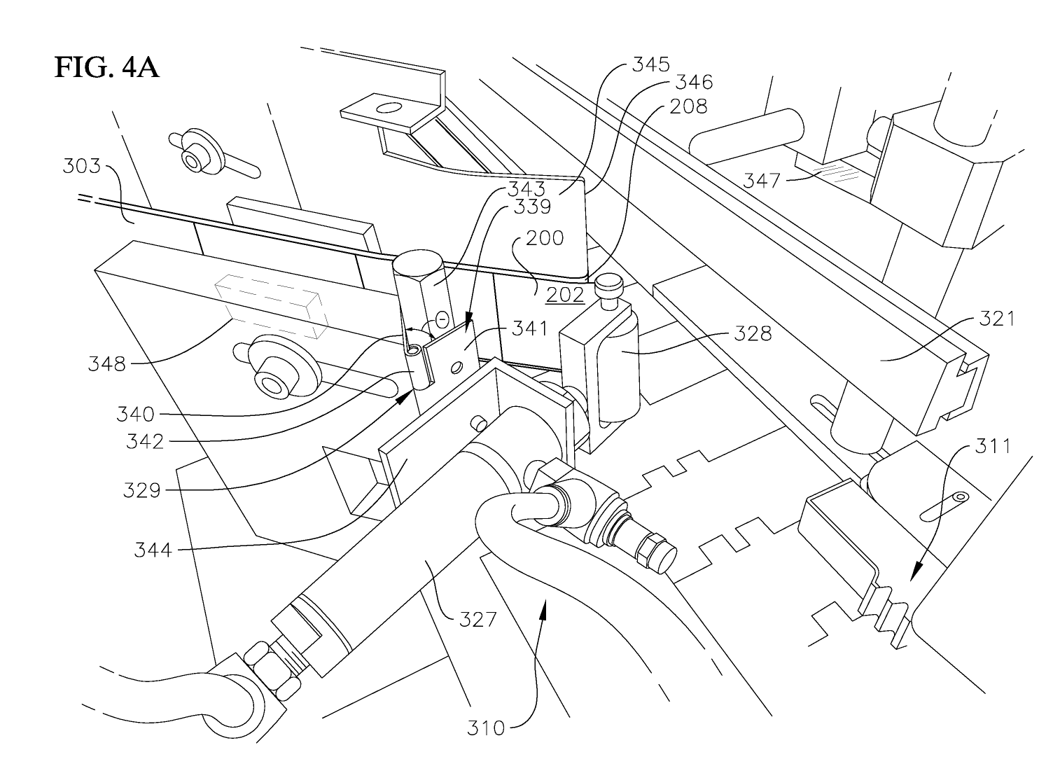

With reference now to the embodiment illustrated in FIGS. 3A-3B, each of the spindle assemblies 304 includes a spindle 312 and a bushing 313. The spindle 313 is fixedly coupled to the movable conveyor track 302 and the spindle 312 is rotatably supported (arrow 314) on the bushing 313. In the illustrated embodiment, the spindle 312 includes a spur gear 315 and a support post 316 extending up from the spur gear 315. In the illustrated embodiment, the spur gear 315 is oriented horizontally such that a rotational axis A of the spur gear 315 is perpendicular or substantially perpendicular to the movable conveyor track 302. Additionally, in the illustrated embodiment, a central axis of the support post 316 is coaxial with the rotational axis A of the spur gear 315. The spindle 312 defines a central axial opening 317 (e.g., a through hole) extending through the spur gear 315 and the support post 316 and extending along the rotational axis A of the spur gear 315.

In the illustrated embodiment, the bushing 313 extends up through the central axial opening 317 in the spindle 312 and is secured to the spindle 312 by a lock spring ring 318 engaging an upper end of the bushing 313 that extends out of the central axial opening 317 in the spindle 312. The lock spring ring 318 is configured to permit the spindle 312 to rotate (arrow 314) relative to the bushing but prevent the spindle 312 from inadvertently sliding vertically off of the bushing (e.g., when the labeled actuators 100 of the metered dose inhalers are lifted off of the spindle assemblies 304). In the illustrated embodiment, a lower end of the bushing 312 includes external threads 319 configured to thread into internal threads 320 in the movable conveyor track 302. In one or more embodiments, the internal threads 320 may be provided in an insert in the movable conveyor track 302. In one or more embodiments, the bushing 312 may be fixedly coupled to the movable conveyor track 302 in any other suitable manner. In one or more embodiments, the spindles 312 may be rotatably coupled (arrow 314) to the movable conveyor track 302 in any other suitable manner (e.g., the spindles 312 may be rotatably supported on rotary bearings coupled to the movable conveyor track 302).

The support post 316 of the spindle 312 is configured to support the actuator 100 of the metered dose inhaler. In the illustrated embodiment, the actuators 100 of the metered dose inhalers are loaded onto the spindles 312 upside down. In the illustrated embodiment, the support post 316 of the spindle 312 is configured to extend up through the opening 104 in the actuator body 101 and extend into the cavity 102 of the actuator body 101. The support post 316 of the spindle 312 may have any shape suitable for the configuration of the product containers that the support post 316 is configured to support (e.g., the support post 316 of the spindle 312 may be configured to conform or substantially conform to at least a portion of the cavity 102 in the actuator 100 of the metered dose inhaler). Additionally, in the illustrated embodiment, when the actuator 100 is supported on the spindle 312, the upper edge 105 of the actuator body 101 is supported on an upper surface of the spur gear 315 such that the spur gear 315 remains exposed (e.g., the spur gear 315 is not covered by the actuator 100 supported on the support post 316).

Additionally, in the illustrated embodiment, the support post 316 of the spindle 312 is configured to support the actuator body 101 of the actuator 100 of the metered dose inhaler perpendicular or substantially perpendicular to the movable conveyor track 302. (e.g., the support post 316 of the spindle 312 is configured to maintain the actuator body 101 in a vertical or substantially vertical orientation). Maintaining the actuator 100 of the metered dose inhaler perpendicular or substantially perpendicular to the movable conveyor track 302 is configured to ensure that the label 200 is applied to the actuator 100 in the desired orientation. For instance, in the illustrated embodiment, the spindle 312 is configured to maintain the actuator 100 of the metered dose inhaler in a vertical orientation such that when the label 200 is applied to the actuator 100 by the actuator assembly 310 and the winding assembly 311, the upper and lower edges 205, 206 of the label 200 are parallel or substantially parallel with the upper edge 105 of the actuator body 101, as illustrated in FIGS. 1A-1B. Otherwise, misorientation between the label 200 and the actuator 100 (e.g., misalignment such that the upper and lower edges 205, 206 of the label 200 are anti-parallel to the upper edge 105 of the actuator body 101) tends to cause wrinkles and/or bubbles in the label 200.

With reference again now to the embodiment illustrated in FIG. 2, the labelling system 300 includes a pair of rails 321, 322 positioned above the movable conveyor track 302. In the illustrated embodiment, the rails 321, 322 are supported on stanchions 323, 324, respectively, coupled to opposite sides of the conveyor 301. In one or more embodiments, the rails 321, 322 are vertically and horizontally adjustable on the stanchions 323, 324, respectively, such that the vertical distance between rails 321, 322 and the movable conveyor track 302 may be adjusted and the horizontal or lateral spacing between the rails 321, 322 may be adjusted to accommodate the size and configuration of the product containers (e.g., the actuators 100 of the metered dose inhalers) that will be labeled utilizing the labelling system 300. The rails 321, 322 extend from a position distal to the applicator arm 303 (e.g., a position proximate to the infeed station 306 of the conveyor 301) to a position proximate to the applicator arm 303. In the illustrated embodiment, one of the rails 321 extends a greater distance toward the outfeed station 307 of the conveyor 301 than the other rail 322. In the illustrated embodiment, the rail 322 proximate to the same side of the conveyor 301 as the applicator arm 303 terminates before the actuator assembly 310. Additionally, in the illustrated embodiment, the rail 321 proximate to the side of the conveyor 301 opposite to the side of the conveyor 301 along which the applicator arm 303 is positioned extends past the applicator arm 303 and the actuator assembly 310.

The rails 321, 322 are configured to maintain the product containers in the desired angular orientation as the movable conveyor track 302 advances (arrow 309) the spindle assemblies 304 and the product containers on the spindle assemblies 304 toward the applicator arm 303 and the actuator assembly 310, which is configured to ensure that the labels are applied with the desired orientation onto the product containers. In one or more embodiments, the lateral distance between the rails 321, 322 may be less than the longest lateral dimension of the product container (e.g., the distance between the rails 321, 322 may be equal or substantially equal to the narrow lateral dimension of the product container) such that the rails 321, 322 prevent the product containers and the spindles 312 from rotating (arrow 314) while positioned between the rails 321, 322. In the illustrated embodiment, the vertical distance between the rails 321, 322 and the movable conveyor track 302 and the horizontal distance between the rails 321, 322 is selected such that the rails 321, 322 are configured to contact opposing sides 111, 112 of the mouth pieces 103 of the actuators 100. Contact between the rails 321, 322 and the opposing sides 111, 112 of the mouth pieces 103 of the actuators 100 is configured to prevent the actuators 100 and the spindles 312 from rotating (arrow 314) while positioned between the rails 321, 322. In the illustrated embodiment, contact between the rails 321, 322 and the sides 111, 112 of the mouth pieces 103 is also configured to orient the actuators 100 such that the mouth pieces 103 of the actuators 100 are oriented lengthwise along the conveyor 301 and back surfaces 107 of the actuator bodies 101 face the applicator arm 303 and the actuator assembly 310. Rotationally orienting the actuators 100 with the rails 321, 322 is configured to ensure that the labels 200 have the desired orientation on the actuators 100 (e.g., rotationally orienting the actuators 100 with the rails 321, 322 is configured to ensure the leading edge portion 204 of the label 200 is applied to the actuator 100 by the actuator assembly 310 is aligned with a laterally centered portion of the back surface 107 of the actuator body 101 of the metered dose inhaler).

In one or more embodiments, the rails 321, 322 may be utilized only when the product container (e.g., the actuator body 101 of the metered dose inhaler) is asymmetric. The pair of rails 321, 322 may be spaced apart from each by any horizontal distance and spaced apart from the movable conveyor track 302 by any vertical distance suitable for the type of product container the labelling system 300 is configured to label and/or the desired orientation of the label on the product container.

Additionally, in the illustrated embodiment, distal ends 325, 326 of the rails 321, 322, respectively, proximate to the infeed station 306 of the conveyor 301 are flared outward away from each other such that the horizontal spacing between the rails 321, 322 at the distal ends 325, 326 is greater than the spacing between the rails 321, 322 along a remaining portion of the rails 321, 322 (e.g., the horizontal spacing between the rails 321, 322 at the distal ends 325, 326 is greater than the horizontal spacing between the rails 321, 322 at intermediate portions and/or proximal ends of the rails 321, 322). The outwardly flared distal ends 325, 326 of the rails 321, 322 are configured to enable misoriented product containers to enter between the rails 321, 322 and reorient the product containers into the desired orientation. For instance, when a misoriented actuator 100 of a metered dose inhaler is supported on one of the spindles 312 and the movable conveyor track 302 is actuated to advance the spindle 312 and the actuator 100 toward the applicator arm 303, at least one of the rails 321, 322 is configured to contact at least one of the sides 111, 112 of the mouth piece 103 of the actuator 100 and thereby rotate the actuator 100 of the metered dose inhaler such that the mouth piece 103 of the actuator 100 is oriented lengthwise along the conveyor 301 (e.g., the sides 111, 112 of the mouth piece 103 are parallel or substantially parallel with the rails 321, 322). In this manner, the rails 321, 322 are configured to both orient the actuators 100 into the desired angular orientation and maintain the product actuators 100 in the desired orientation until the actuators 100 reach the applicator arm 303 and the leading edge 204 of the label 200 is applied to the actuators 100 by the actuator assembly 310.

Additionally, the rail 321 that extends past the applicator arm 303 and the actuator assembly 310 is configured to maintain contact with the actuator 100 (e.g., one of the sides 111, 112 of the mouth piece 103) as the actuator assembly 310 applies the leading edge 204 of the label 200 to the actuator 100. In this manner, the rail 321 is configured to prevent the actuator 100 from rotating as the actuator assembly 310 applies the leading edge 204 of the label 200 to the actuator 100, which might otherwise cause misapplication of the label 200 to the actuator 100.

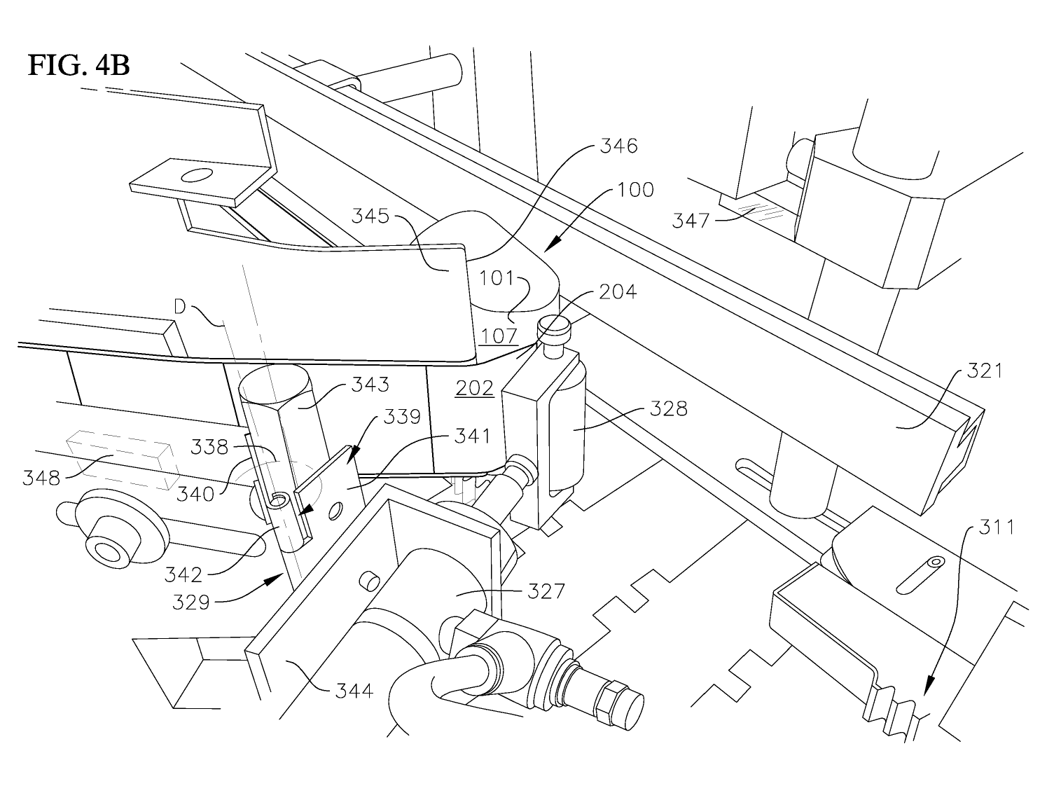

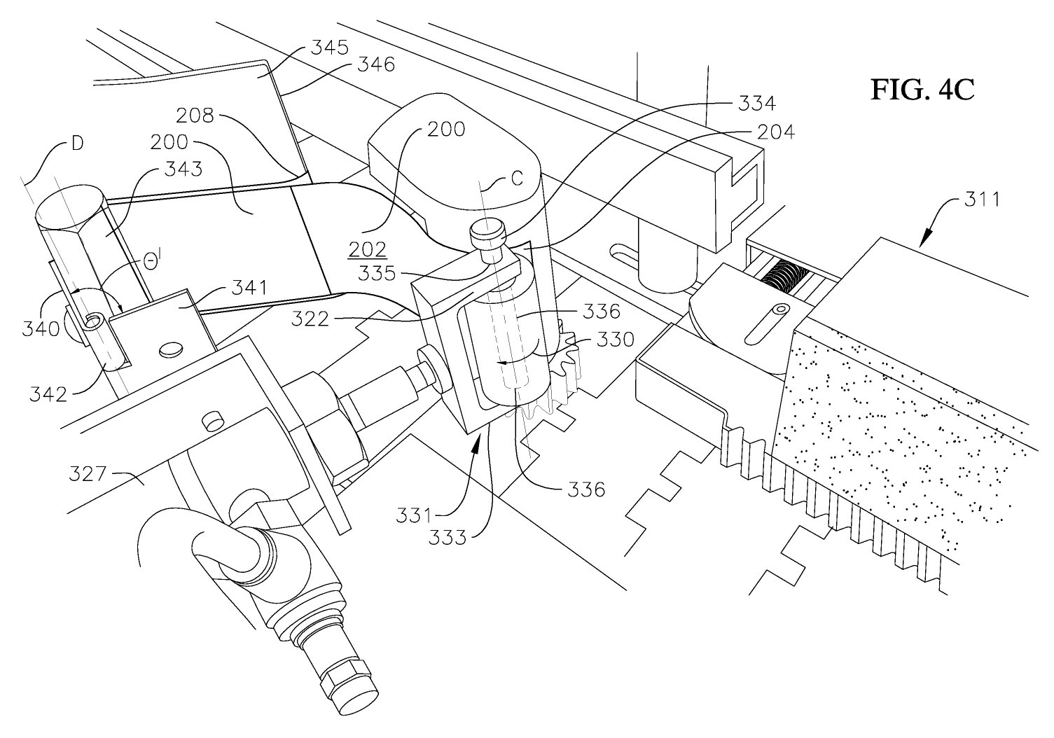

With reference now to the embodiment illustrated in FIGS. 4A-4C, the actuator assembly 310 includes an actuator 327, a roller 328 rotatably coupled to an end of the actuator 327 proximate to the conveyor 301, and a hinge assembly 329 coupled to the actuator 327. The actuator 327 is configured to move between a retracted position (FIG. 4A) and an extended position (FIG. 4B). The actuator 327 may be any suitable type of actuator, such as a pneumatic cylinder, a hydraulic cylinder, or an electromechanical cylinder. In the illustrated embodiment, the actuator 327 is positioned downstream of the applicator arm 303 (e.g., the actuator 327 is positioned between the applicator arm 303 and the wrapping assembly 311). In the illustrated embodiment, when the actuator 327 is in the extended position (FIG. 4B), the roller 328, which is coupled to the end of the actuator 327, is aligned or substantially aligned with the actuator 100 supported on the spindle assembly 304. When the actuator 327 is in the extended position, the roller 328 is configured to press the leading edge portion 204 of the label 200 against the actuator body 101 of the metered dose inhaler (e.g., the roller 328 is configured to press the leading edge portion 204 of the label 200 against the back surface 107 of the actuator body 101).

In the illustrated embodiment, the roller 328 is rotatably (arrow 330) coupled to the end of the actuator 327 by a knuckle or a fork 331 including a first arm 332 and a second arm 333 spaced apart from the first arm 332. The roller 328 extends between the arms 332, 333 of the knuckle 331 and is rotatably coupled to the knuckle 331 by a pin 334 extending through coaxial openings 335, 336 in the arms 332, 333, respectively, and a central axial opening 337 of the roller 328. In the illustrated embodiment, the roller 328 is configured to rotate (arrow 330) about an axis C that is orthogonal or substantially orthogonal to the movable conveyor track 302 (e.g., a vertical or substantially vertical axis). The roller 328 is configured to rotate (arrow 330) about the axis C of the roller 328 when the roller 328 contacts the actuator 100 to apply and/or wrap a portion of the label 200 around at least a portion of the actuator body 101. In one or more embodiments, an exterior surface of the roller 328 may include a resilient material, such as foam. As described in more detail below, the resilient material of the roller 328 allows the roller 328 to conform to non-cylindrical surfaces (e.g., a rotationally asymmetric surface) of the product container. In the illustrated embodiment, the resilient material of the roller 328 is configured to compress and conform to a portion of the back surface 107 of the actuator body 101 when applying the label 200 to the actuator body 101, which is configured to prevent the formation of wrinkles and bubbles in the label 200.

With continued reference to the embodiment illustrated in FIGS. 4A-4C, the hinge assembly 329 is configured to permit the actuator 327 and the roller 328 to rotate (e.g., swing) (arrow 338) about an axis D that is orthogonal or substantially orthogonal to the movable conveyor track 302 (e.g., the roller 328 and the actuator 327 are configured to swing (arrow 338) about a vertical or substantially vertical axis). The actuator 327 and the roller 328 coupled to the end of the actuator 327 are configured to swing (arrow 338) between a rearward position (FIGS. 4A-4B) and a forward position (FIG. 4C). The rearward position is the neutral position of the actuator 327 and the roller 328.

As described in more detail below, contact between the roller 338 and the actuator 100 during application of the leading edge portion 204 of the label 200 to the actuator 100 is configured swing (arrow 338) the actuator 327 and the roller 328 about the axis D from the rearward position to the forward position (e.g., contact between the roller 338 and the actuator 100 during application of the leading edge portion 204 of the label 200 to the actuator 100 is configured swing (arrow 338) the actuator 327 and the roller 328 about the axis D in the direction in which the movable conveyor track 302 is advancing (arrow 309 in FIG. 2) the spindle assembly 304 and the actuator 100 supported on the spindle assembly 304). Moreover, the swinging (arrow 338) of the actuator 327 and the roller 328 about the axis D is configured to enable the roller 328 to wrap a portion of the label 200 around at least a portion of a back surface 107 of the actuator body 101 of the metered dose inhaler.

With continued reference to the embodiment illustrated in FIGS. 4A-4C, the hinge assembly 329 includes a spring-loaded hinge 339 configured to bias the actuator 327 and the roller 328 into the rearward position (e.g., the spring-loaded hinge 339 is configured to bias the actuator 327 and the roller 328 to swing (arrow 338) in a direction opposite to the direction in which the movable conveyor track 302 is advancing the spindle assembly 304 and the actuator 100). As described in more detail below, the spring-loaded hinge 339 is configured to return the actuator 327 and the roller 328 to the rearward position after the roller 328 has applied the leading edge portion 204 of the label 200 to the actuator 100 and the roller 328 has disengaged the actuator 100. In the illustrated embodiment, the spring-loaded hinge 339 includes a first hinge leave 340 and a second hinge leave 341 hingedly coupled to the first hinge leave 340 by a hinge 342. In the illustrated embodiment, the first hinge leave 340 is fixedly coupled to a fixture 343 (e.g., a support post coupled to a side of the conveyor 301) and the second hinge leave 341 is coupled to a bracket 344 coupled to the actuator 327. The second hinge leave 341 is configured to rotate (arrow 338) about the hinge 342 toward and away from the first hinge leave 340 (e.g., the second hinge leave 341 is configured to rotate about the hinge 342 between a first angular position in which the second hinge leave 341 is spaced apart from the first hinge leave 340 by a first angle θ and a second angular position in which the second hinge leave 341 is spaced apart from the first hinge leave 340 by a second angle θ′ greater than the first angle θ). The rotation of the second hinge leave 341 relative to the first hinge leave 340 is configured to swing (arrow 338) the actuator 327 and the roller 328 connected to the end of the actuator 327 about the axis D. Additionally, in the illustrated embodiment, the spring-loaded hinge 339 includes at least one spring contacting the first and second hinge leaves 340, 341. The spring is configured to bias the second hinge leave 341 into the first angular position and thereby bias the actuator 327 and the roller 328 into the rearward position.

In one or more embodiments, the hinge assembly 329 may be provided without the spring-loaded hinge 339 and the hinge assembly 329 may include any other suitable type of resilient member configured to bias the actuator 327 and the roller 328 into the rearward position (e.g., a spring, such as a torsion spring, an extension spring, or a compression spring).

With continued reference to the embodiment illustrated in FIGS. 4A-4C, the labelling system 300 also includes a label peeler 345 (e.g., a peel plate) configured to separate the labels 200, one by one, from a removable backing sheet 208 on which the labels 200 are provided, thereby exposing the adhesive surface 201 of the label 200. In the illustrated embodiment, the label peeler 345 is positioned at or near an end of the applicator arm 303 proximate to the conveyor 301. In the illustrated embodiment, the labels 200 separate from the backing sheet 208 as the backing sheet 208 wraps around a leading edge 346 of the label peeler 345.

In the illustrated embodiment, the labelling system 300 includes a product container sensor 347 (e.g., a proximity sensor, such as a laser sensor) configured to detect when an actuator 100 supported on one of the spindle assemblies 304 has been advanced by the movable conveyor track 302 to a position proximate to the applicator arm 303. As described in more detail below, when the product container sensor 347 detects a actuator 100 on one of the spindle assemblies 304 proximate to the applicator arm 303, a label 200 is fed along the applicator arm 303 toward the conveyor 301 and the label peeler 345 separates the label 200 from the removable backing sheet 208.

With continued reference to the embodiment illustrated in FIGS. 4A-4C, the labelling system 300 also includes a static control bar 348 (e.g., an anti-static gun) (see also FIG. 2). In the illustrated embodiment, the static control bar 348 is positioned proximate to the end of the applicator arm 303 proximate to the conveyor 301. Additionally, in the illustrated embodiment, when the applicator arm 303 advances a label 200 toward the conveyor 301, the label peeler 345 and the static control bar 348 are on opposite sides of the label 200 (e.g., the applicator arm 303 advances the label 200 between the label peeler 345 and the static control bar 348). The static control bar 348 is configured to mitigate the buildup of static on the labels 200, which might otherwise cause the labels 200 to curl. Additionally, the curling of the labels 200 due to the buildup of static on the labels 200 may inhibit proper application of the labels 200 to the product containers (e.g., the actuators 100 of the metered dose inhalers).

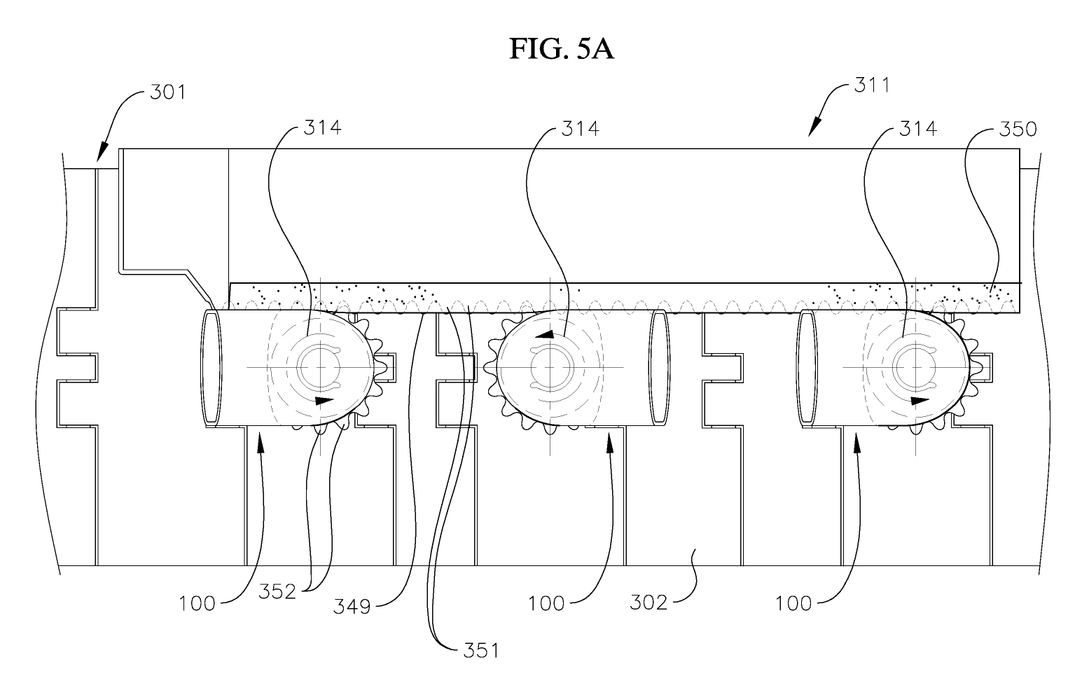

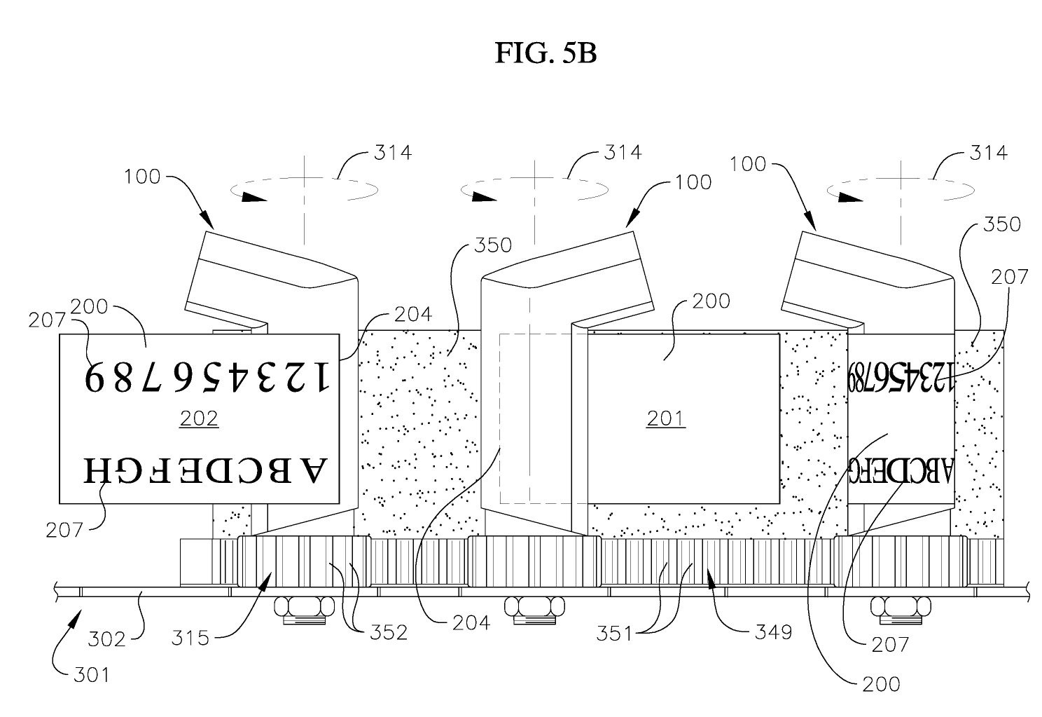

With reference now to the embodiment illustrated in FIGS. 5A-5B, the wrapping assembly 311 includes a gear rack 349 and an applicator plate 350. In the illustrated embodiment, the gear rack 349 and the applicator plate 350 are positioned along a side of the conveyor 301 opposite to the actuator assembly 310. In the illustrated embodiment, the gear rack 349 has a series of teeth 351. Additionally, in the illustrated embodiment, the gear rack 349 is a straight or substantially straight segment (e.g., the gear rack 349 is a linear or substantially linear segment). The teeth 351 of the gear rack 349 face inward toward a centerline of the movable conveyor track 302. The teeth 351 of the gear rack 349 are configured to mesh with teeth 352 of the spur gear 315 of the spindle. As described in more detail below, the engagement between the teeth 352 of the spur gear 315 of the spindle and the teeth 351 of the gear rack 349 is configured to rotate (arrow 353) the spindle 312 and the actuator 100 around the bushing 313 about the rotational axis A of the spur gear 315 to wrap a remaining portion of the label 200 around the actuator body 101 of the actuator 100.

With continued reference to the embodiment illustrated in FIGS. 5A-5B, the applicator plate 350 is positioned along at least a portion of the gear rack 349 (e.g., the applicator plate 350 overlaps at least a portion of the gear rack 349). In the illustrated embodiment, the applicator plate 350 extends above the gear rack 349 (e.g., the applicator plate 350 extends upward away from the movable conveyor track 302). In the illustrated embodiment, the applicator plate 350 includes a layer 354 of resilient and compressible material (e.g., foam or rubber, such as ethylene propylene diene monomer (EPDM)) facing toward a centerline of the movable conveyor track 302. As described in more detail below, the resilient and compressible layer 354 is configured to engage the actuator 100 as the spindle 312 and the actuator 100 are rotated (arrow 353) due to the engagement between the teeth 352, 351 of the spur gear 315 and the gear rack 349. The resilient and compressible layer 354 allows the applicator plate 350 to conform to the shape of a non-cylindrical product container (e.g., the back and front surfaces 107, 108 of the actuator body 101 of the metered dose inhaler), which is configured to prevent the formation of wrinkles and bubbles in the label 200.

In operation, the conveyor motor 308 actuates the movable conveyor track 302 to advance the movable conveyor track 302, the spindle assemblies 304 coupled to the movable conveyor track 302, and the product containers (e.g., the actuators 100 of the metered dose inhalers) supported on the spindle assemblies 304 between the pair of rails 321, 322 toward the applicator arm 303. The product containers may be loaded (e.g., manually loaded) on the spindle assemblies 304 proximate to the infeed station 306 during and/or before actuation of the movable conveyor track 302. For instance, in one or more embodiments in which the product containers are actuators 100 of metered dose inhalers, each of the actuators 100 may be loaded onto one of the spindles 312 by turning the actuator 100 upside down and sliding the actuator 100 over the spindle 312 such that the spindle 312 extends up through the opening 104 and into the cavity 102 of the actuator body 101. As illustrated in FIG. 2B, the spindles 312 are configured to support the actuator bodies 101 of the actuators 100 perpendicular or substantially perpendicular to the movable conveyor track 302.

As described above, the rails 321, 322 are configured to maintain the product containers in the desired angular orientation as the product containers are advanced toward the applicator arm 303 by the movable conveyor track 302. For instance, in one or more embodiments in which the product containers are actuators 100 of metered dose inhalers, each of the actuators 100 of the metered dose inhalers may be loaded onto the spindles 312 with the back surface 107 of the actuator 100 facing the applicator arm 303 (e.g., the back surface 107 of the actuator 100 facing the outfeed station 307 of the conveyor 301), and the rails 321, 322 engage the opposing sides 111, 112 of the mouth piece 103 of the actuator 100 and thereby maintain this orientation of the actuator 100 until the leading edge portion 204 of the label 200 is applied to the actuator 100 by the actuator assembly 310. In this manner, the rails 321, 322 are configured to ensure that the labels 200 are applied to the product containers with the desired orientation (e.g., the leading edge portion 204 of the label 200 is centered on the back surface 107 of the actuator body 101 such that the indicia 207 on the label 200 has the desired orientation on the actuator body 101).

When the product container sensor 347 of the labelling system 300 detects that the movable conveyor track 302 has advanced (arrow 309) the product container to a position proximate to the applicator arm 303, the applicator arm 303 advances one of the labels 200 from the spool of labels supported on a label reel 305 along the applicator arm 303 toward the end of the applicator arm 303 proximate to the conveyor 301. Once the label 200 reaches the end of the applicator arm 303 proximate to the conveyor 301, the label peeler 345 peels the leading edge portion 204 of the label 200 off of the backing sheet 208, thereby exposing the adhesive surface 201 of the label 200. In the illustrated embodiment, once the leading edge portion 204 of the label 200 has been peeled off of the backing sheet 208 by the label peeler 345, the adhesive surface 201 of the label 200 faces the advancing product container and the non-adhesive surface 202 of the label 200 bearing the indicia 207 faces the roller 328 and the actuator 327 (e.g., the non-adhesive surface 202 of the label 200 bearing the indicia 207 faces away from the advancing product container).

When one of the spindle assemblies 304 and the product container supported thereon are sufficiently close to the applicator arm 327, as detected by the product container sensor 347, the actuator 327 moves into the extended position. As the actuator 327 moves into the extended position, the roller 328 connected to the end of the actuator 327 engages the non-adhesive surface 202 of the label 200 bearing the indicia 207 and presses the adhesive surface 201 of the leading edge portion 204 of the label 200 against the product container (e.g., the extension of the actuator 327 adheres leading edge portion 204 of the label 200 against the back surface 107 of the actuator body 101 of the metered dose inhaler).

When the actuator 327 is in the extended position and the roller 328 is in contact with the product container, the continued advancement arrow 309) of the spindle assembly 304 and the product container supported thereon in the direction of the outfeed station 307 by the movable conveyor track 302 causes the roller 328 and the actuator 327 to swing (arrow 338) about the axis D of the hinge assembly 329 in the forward direction toward the outfeed station 307 (e.g., the contact between the advancing product container and the roller 328 swings (arrow 338) the roller 328 and the actuator 327 in the direction in which the movable conveyor track 302 is advancing the spindle assembly 304 and the product container).

Once the roller 328 has applied the leading edge portion 204 of the label 200 to the product container, the actuator 327 retracts toward the retracted position. Once the actuator 327 has sufficiently retracted such that the roller 328 clears the product container, the spring-loaded hinge 339 of the hinge assembly 329 forces the actuator 327 and the roller 328 to swing (arrow 338) in the direction opposite to the direction in which the movable conveyor track 302 is advancing (arrow 309) the spindle assembly 304 and the product container (e.g., the spring-loaded hinge 339 of the hinge assembly 329 is configured to swing the actuator 327 and the roller 328 rearward toward the infeed station 306 of the conveyor 301). As the actuator 327 and the roller 328 swing (arrow 338) rearward, contact between the product container and the roller 328 rotates (arrow 330) the roller 328 about its axis C. As the roller 328 rotates (arrow 330), the roller 328 follows along a portion of the product container (e.g., a portion of the back surface 107 of the actuator body 101 of the metered dose inhaler) and applies (e.g., presses) the label 200 along this portion of the product container.

Once the movable conveyor track 302 has advanced (arrow 309) the spindle assembly 304 and the product container past the applicator arm 327 and the roller 328, the teeth 352 of the spur gear 315 engage the teeth 351 of the gear rack 349 of the winding assembly 311. The engagement between the teeth 352, 351 of the spur gear 315 and the gear rack 349 spins (arrow 353) the spindle 312 and the product container supported on the spindle 312 about the rotational axis A (e.g., the vertical axis) of the spur gear 315 (e.g., the engagement between the spur gear 315 and the gear rack 349 is configured to rotate (arrow 353) the spindle 312 and the product container around the bushing 313 coupled to the movable conveyor track 302).

As the spindle 312 and the product container spin (arrow 353), the resilient and compressible layer 354 of the applicator plate 350 presses and wraps a remaining portion of the label 200 around the product container (e.g., the resilient and compressible layer 354 the applicator plate 350 presses and wraps a remaining portion of the label 200 around the back and front surfaces 107, 108 of the actuator body 101 of the metered dose inhaler). The resilient and compressible layer 354 allows the applicator plate 350 to conform to the shape of a non-cylindrical product container (e.g., the rotationally asymmetric back and front surfaces 107, 108 of the actuator body 101 of the metered dose inhaler). In this manner, the resilient and compressible layer 354 allows the applicator plate 350 to maintain continuous contact between the label 200 and the product container as the product container is spinning (arrow 353), which is configured to prevent the formation of wrinkles and/or bubbles in the label 200. In one or more embodiments, the spinning (arrow 353) of the spindle 312 and the product container causes an entirety of the surface or surfaces along which the label 200 is to be applied to contact the applicator plate 350. In one or more embodiments, the length of the gear rack 349 and the applicator plate 350 may be configured to cause the spindle and the product container to rotate (arrow 353) at least one complete revolution (i.e., 360 degrees or more) in contact with the applicator plate 350. In one or more embodiments, the length of the gear rack 349 and the applicator plate 350 may be configured to cause the spindle 312 and the product container to rotate (arrow 353) more than one complete revolution (e.g., approximately 720 degrees or more) in contact with the applicator plate 350. In one or more embodiments, the labelling system 300 and the label 200 are configured such that the ends 203, 204 of the label 200 overlap once the label 200 is applied to the product container (see FIGS. 1A-1B).

Once the movable conveyor track 302 has the advanced (arrow 309) the spindle assembly 304 and the labeled product container past the winding assembly 311 and to the outfeed station 307 of the conveyor 301, the labeled product container (e.g., the labeled actuator 100 of the metered dose inhaler) may be removed from the spindle 312 (e.g., the labeled product container may be manually lifted off of the spindle 312).

While this invention has been described in detail with particular references to embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention. Although relative terms such as “horizontal,” “vertical,” “upper,” “lower,” “inner,” “outer” and similar terms have been used herein to describe a spatial relationship of one element to another, it is understood that these terms are intended to encompass different orientations of the various elements and components of the invention in addition to the orientation depicted in the figures. Additionally, as used herein, the term “substantially” and similar terms are used as terms of approximation and not as terms of degree, and are intended to account for the inherent deviations in measured or calculated values that would be recognized by those of ordinary skill in the art. Moreover, the tasks described above may be performed in the order described or in any other suitable sequence. Additionally, the methods described above are not limited to the tasks described. Instead, for each embodiment, one or more of the tasks described above may be absent and/or additional tasks may be performed. Furthermore, as used herein, when a component is referred to as being “on” or “coupled to” another component, it can be directly on or attached to the other component or intervening components may be present therebetween.

What is claimed is:

1. A labelling system configured to apply a label to a product container, the labelling system comprising:
a conveyor comprising a movable track, the conveyor having an infeed station and an outfeed station opposite the infeed station;
a spindle rotatably supported on the movable track and configured to support the product container;
an applicator arm configured to feed the label toward the conveyor when the movable track advances the spindle and the product container toward the applicator arm;
an actuator configured to move between a retracted position and an extended position; and
a roller rotatably coupled to an end of the actuator, wherein the roller is configured to press a leading edge of the label against the product container when the actuator is in the extended position, and wherein the roller is configured to rotate and follow along at least a portion of the product container and press the label against the portion of the product container.

2. The labelling system of claim 1, wherein the actuator is configured to swing in a first direction about an axis substantially perpendicular to the movable track.

3. The labelling system of claim 2, further comprising a spring hinge coupled to the actuator, the spring hinge configured to bias the actuator to swing in a second direction opposite the first direction.

4. The labelling system of claim 1, wherein an outer surface of the roller comprises a resilient material.

5. The labelling system of claim 4, wherein the resilient material of the roller is foam.

6. The labelling system of claim 1, wherein the actuator is a hydraulic cylinder.

7. The labelling system of claim 1, wherein the actuator is a pneumatic cylinder.

8. The labelling system of claim 1, further comprising a pair of rails supported above the movable track, wherein the pair of rails extends from a first end proximate to the infeed station of the conveyor and a second end proximate to the applicator arm.

9. The labelling system of claim 1, further comprising a peel plate proximate to an end of the applicator arm proximate to the conveyor, wherein the peel plate is configured to peel the label off of a removable backing sheet.

10. The labelling system of claim 9, further comprising an anti-static bar proximate to the end of the applicator arm.

11. A labelling system configured to apply a label to a product container, the labelling system comprising:
a conveyor comprising a movable track, the conveyor having an infeed station and an outfeed station opposite the infeed station;
a spindle rotatably supported on the movable track, the spindle comprising a spur gear and a support post configured to support the product container;
an applicator arm configured to feed the label toward the conveyor when the movable track advances the spindle and the product container toward the applicator arm;
an actuator configured to move between a retracted position and an extended position, wherein the actuator in the extended position is configured to press a leading edge of the label against the product container;
a gear rack positioned along a side of the conveyor; and
an applicator plate positioned along at least a portion of the gear rack, the applicator plate comprising a resilient material facing a centerline of the conveyor,
wherein engagement between the spur gear and the gear rack is configured to rotate the spindle and the product container, and
wherein the resilient material of the applicator plate is configured to press a remaining portion of the label onto the product container during the engagement between the spur gear and the gear rack.

12. The labelling system of claim 11, wherein the resilient material of the applicator plate comprises ethylene propylene diene monomer.

13. The labelling system of claim 11, wherein the gear rack is substantially straight.

14. The labelling system of claim 13, wherein the applicator plate is substantially planar.

15. The labelling system of claim 11, wherein the gear rack is configured to rotate the spindle and the product container at least one complete revolution along the applicator plate.

16. The labelling system of claim 11, further comprising a roller rotatably coupled to an end of the actuator.

17. The labelling system of claim 11, further comprising a pair of rails supported above the movable track, wherein the pair of rails extends from a first end proximate to the infeed station of the conveyor and a second end proximate to the applicator arm.

18. The labelling system of claim 11, further comprising a peel plate proximate to an end of the applicator arm proximate to the conveyor, wherein the peel plate is configured to peel the label off of a removable backing sheet.

19. The labelling system of claim 18, further comprising an anti-static bar proximate to the end of the applicator arm.
